(19) United States
(12) Patent Application Publication
(10) Pub. No.: US 2009/0247785 A1
(43) Pub. Date: Oct. 1, 2009

(54) METHODS AND SYSTEMS FOR PRETREATMENT OF AN OIL STREAM

(75) Inventors: Richard S. Parnas, West Hartford, CT (US); Matthew B. Boucher, Somerville, MA (US)

Correspondence Address: CANTOR COLBURN, LLP, 20 Church Street, 22nd Floor, Hartford, CT, 06103, US

(73) Assignee: THE UNIVERSITY OF CONNECTICUT, Farmington, CT (US)

(21) Appl. No.: 12/415,351

(22) Filed: Mar. 31, 2009

Related U.S. Application Data
(60) Provisional application No. 61/040,908, filed on Mar. 31, 2008.

(51) Int. Cl. C07C 53/50; B01J 19/00

(52) U.S. Cl. 562/512; 422/225

(57) ABSTRACT

In one embodiment, a method for pre-treating an oil comprises: introducing a oil, an alcohol, and an acid catalyst to a first continuous stirred tank reactor vessel, wherein the oil comprises free fatty acids; selectively reacting the oil and the alcohol to convert the free fatty acids to alkyl esters, wherein the reaction does not produce glycerol; removing a pre-treated oil stream from a lower portion of the first continuous stirred tank reactor, wherein the pre-treated oil stream comprises the alkyl esters; and recycling the alcohol and acid catalyst from an upper portion of the first continuous stirred tank reactor.

### CROSS-REFERENCE TO RELATED APPLICATIONS

This application claims the benefit of the filing date of U.S. Patent Application Ser. No. 61/040,908 filed Mar. 31, 2008, which is hereby incorporated by reference.

### TECHNICAL FIELD

This application relates to pretreatment of oil and grease, and especially relates to the continuous removal of free fatty acids from oil and grease streams.

### BACKGROUND

Global concerns regarding greenhouse gas emissions combined with soaring oil prices have driven the search for renewable diesel fuels derived from either virgin or waste vegetable oils, known as “biodiesels.” One potential solution to mitigate environmental and macroeconomic displacements is the use of energy derived from locally produced biomass. Two particular such bio-fuels are ethanol and biodiesel. While ethanol has garnered more attention due to a longer track record and the activities in Brazil, biodiesel is actually a higher energy fuel with greater environmental benefits. Typically biodiesel is produced by transesterification of triglycerides with methanol.

Biodiesel fuels can replace petroleum-based diesel fuels with minimum modifications to existing diesel engines, oil heating systems and fuels infrastructure. Biodiesel is non-toxic and biodegradable resulting in less harmful emissions. Biodiesel is currently produced via transesterification of triglycerides (TG) with an alcohol, specifically methanol due to its favorable kinetics. The global transesterification mechanism of TG encompasses three sequential reversible reactions wherein triglycerides (TG) react to form diglycerides (DG), monoglycerides (MG) and final product glycerol (G).

The source of triglycerides for biodiesel production can range from virgin vegetable oils to waste cooking oils, animal fats, and soapstocks. Due to high prices of virgin vegetable oils there is an interest in diversifying feedstock for biodiesel production. Waste cooking oils and animal fats can be used to produce biodiesel; however, they can contain a considerable amount of free fatty acid (FFA). The chemical structure of FFA is a hydrocarbon chain (C₁₄-C₂₂ with 1-3 double bonds), extending from the carboxylic acid group. In base catalyzed transesterification of vegetable oils, FFA will react with the base catalyst to form soap. Soap formation can cause considerable loss in yield during biodiesel purification steps by emulsion.

Additionally, the acid value or acid content of biodiesel must meet specifications denoted by ASTM method 644-04 to be considered ASTM quality. Any considerable amount of FFA will prevent biodiesel from meeting ASTM standards. In the interest of producing biodiesel, free fatty acids can be removed from waste cooking oils and animal fats via esterification with methanol and catalyst.

Most of the production of biodiesel fuel is carried out in batch reactors, where measured quantities of the triglycerides, methanol, and catalyst are added to a tank, heated, and mixed for a period of time ranging from 1 hour to several hours. After a period of time, the reacted mixture is pumped to another vessel and allowed to sit, quiescent, for a second period of time. The mixture then phase separates into a biodiesel layer and a glycerol layer, and the glycerol layer is drained. The resulting biodiesel is then further purified.

Waste vegetable oils and greases commonly contain free fatty acids (FFA). When the incoming oil contains FFA, processing difficulties can occur due to the surfactant nature of the FFA. Base catalyzed transesterification does not remove FFA. Acid catalysis can be used to remove (i.e., convert) the FFA from the oil. In order to produce methyl or other alkyl esters (i.e., biodiesel) via a base-catalyzed transesterification process, the FFA must be esterified with an alcohol. In one example, the FFA can be converted to biodiesel (methyl esters) by combination with methyl alcohol (methanol) and catalyzed.

Current processes use sulfuric acid to convert the FFA in the oil. Sulfuric acid, however, also causes conversion of the vegetable oil to methyl esters and glycerol. In other current processes, immobilized acid catalysts are used, in which the acid catalyst is immobilized on a small particle. The particles are assembled into a column, through which the oil stream flows. The FFA in the stream is converted to alkyl esters, but after a period of time, the acid catalyst must be regenerated. In both types of processes, the operation costs can be quite expensive, and in most cases the conversion of the FFA is not selective or continuous.

Although these processes successfully convert FFA into alkyl esters, more efficient, selective, continuous, and economical processes are sought.

### BRIEF DESCRIPTION

This disclosure is directed to methods and systems for removing free fatty acids (FFA) from a stream comprising greater than 0.5 mass percent (mass %) FFA.

In one embodiment, a method for pre-treating an oil comprises: introducing the oil, an alcohol, and an acid catalyst to a first continuous stirred tank reactor vessel, wherein the oil comprises free fatty acids; selectively reacting the oil and the alcohol to convert the free fatty acids to alkyl esters; removing a pre-treated oil stream from a lower portion of the first continuous stirred tank reactor, wherein the pre-treated oil stream comprises the alkyl esters; and recycling the alcohol and acid catalyst from an upper portion of the first continuous stirred tank reactor.

In another embodiment the method comprises: combining the oil with an alcohol and an acid catalyst to form a combined liquid stream, wherein the oil comprises free fatty acids; introducing an oil, an alcohol, and an acid catalyst to a continuous stirred tank reactor vessel comprising a first inlet and a first outlet; selectively reacting the oil and the alcohol to convert the free fatty acids to alkyl esters and produce a pre-treated oil, (e.g., wherein the reaction does not produce glycerol); separating the pre-treated oil from the alcohol and acid catalyst in a liquid/liquid separator vessel; wherein the pre-treated oil stream comprises the alkyl esters; removing the pre-treated oil stream from a lower portion of the liquid/liquid separator vessel; and recycling the alcohol and acid catalyst from an upper portion of the liquid/liquid separator vessel to the combined liquid stream.

In one embodiment, a system for pre-treating an oil feed stream, can comprise: a first continuous stirred tank reactor comprising a combined liquid inlet, a first reaction stream outlet, and a first agitator, wherein the combined liquid inlet is in fluid communication with a liquid oil source comprising free fatty acids and a liquid alcohol source comprising an acid catalyst, wherein the free fatty acids are configured to react with the alcohol source and be converted to alkyl esters; and a liquid/liquid separator in fluid communication with the first continuous stirred tank reactor, wherein the separator is configured to separate the alkyl esters and the oil from the alcohol and the acid catalyst, wherein the separator comprises a separator inlet, a pre-treated oil outlet at a lower portion of the separator, and a recycle outlet at an upper portion of the separator, wherein the pre-treated oil outlet is configured to remove the oil and the alkyl esters and the recycle outlet is configured to remove the alcohol and the acid catalyst.

The above described and other features are exemplified by the following figures and detailed description.

### BRIEF DESCRIPTION OF THE DRAWING

Refer now to the figures, which are merely exemplary, not limiting, and wherein like numbers are numbered alike.

FIG. 1 is a schematic illustration of an exemplary embodiment of an oil pre-treatment system comprising four continuous stirred tank reactors in series.

FIG. 2 is a graphic illustration of the reduction in free fatty acid concentration as a function of time by varying reaction temperature for the selective pretreatment process disclosed herein.

FIG. 3 is a graphic illustration of the reduction in free fatty acid concentration as a function of time by varying acid catalyst concentration for the selective pretreatment process disclosed herein.

FIG. 4 is a graphic illustration of the concentration of free fatty acid and bound glycerine for Sample 9.

FIG. 5 is a graphic illustration of the concentration of linoleic acid with time with sulfuric acid as the catalyst at different initial concentrations of linoleic acid.

FIG. 6 is a graphic illustration of the concentration of linoleic acid with time with hydrochloric acid as the catalyst at different initial concentrations of linoleic acid.

FIG. 7 is a graphic illustration of the concentration of linoleic acid with time for esterification catalyzed by sulfuric acid for the initial run compared to its reuse and experiments with initial water concentration.

FIG. 8 is a graphic illustration of the concentration of linoleic acid with time for esterification catalyzed by hydrochloric acid for the initial run compared to its reuse and experiments with initial water concentration.

FIG. 9 is a graphic illustration of the linearization of the initial free fatty acid conversion data when sulfuric acid was used for a catalyst and the initial concentration of free fatty acid was 5 weight percent.

FIG. 10 is a graphic illustration of the linearization of the initial free fatty acid conversion data when hydrochloric acid was used for a catalyst and the initial concentration of free fatty acid was 5 weight percent.

FIG. 11 is a graphic illustration of the sensitivity of sulfuric acid and hydrochloric acid to water.

### DETAILED DESCRIPTION

Disclosed herein is a continuous method of pretreating an oil stream (e.g., natural oils and food grease) by removing free fatty acids (FFA) found in the oil through selectively converting the FFA to methyl esters (i.e., biodiesel fuel). The method achieves conversion of FFA while avoiding further converting the oil to methyl esters and glycerol (e.g., wherein the reaction does not produce glycerol) through the use of a highly selective acid catalyst. The method is further advantageously continuous and adaptive, meaning the process as disclosed herein can be carried out continuously and can adapt with varying levels of FFA in the oil feed streams. Moreover, the method achieves separation of the pretreated oil and the alcohol and acid catalyst by density driven phase separation; the alcohol and acid flowing upward and the pretreated oil settling downward. The reaction can occur in multiple vessels, or the reaction and separation can occur in one single vessel wherein the reaction and separation occur continuously so that the separated pretreated oil continues to emerge from the vessel as long as stream(s) and the alcohol and acid catalyst are fed into the vessel. Moreover, the separated alcohol and acid catalyst can be advantageously recycled.

Also disclosed herein are methods utilizing sulfuric acid and hydrochloric acid as catalysts for the esterification of free fatty acid with methanol in the presence of oil. Further disclosed is catalyst recoverability after esterification at several different initial concentrations of free fatty acid in oil. The effect of water, as a product of esterification, can be determined by reusing a recovered methanol layer for esterification of free fatty acid in the presence of oil. By repeating experiments and changing initial water concentrations, the effect of accumulating water in the methanol layer can be determined.

The method disclosed herein employs an acid catalyzed esterification reaction process that is selective, adaptable to various oil feed stream compositions, and efficient, and can be continuous. Advantageously, the acid catalyst used herein is highly selective in that the FFA are converted to methyl esters without the conversion of the oil to methyl esters and glycerol; thereby creating a highly selective conversion process. The continuous operation can be achieved by carrying out the reaction in a series of continuous stirred tank reactors (CSTRs). The flow path of the feed stream can be controlled in order to use only the minimum number of CSTR in the pretreatment of the stream based on the FFA content therein, thereby improving the efficiency of the system and reducing operation expenses. Even further, the alcohol and acid catalyst used in the esterification reaction can be recycled (e.g., to the first or another CSTR being used in the system), thereby further lowering operation costs by reducing the need for fresh acid catalyst.

As mentioned above, multiple vessels (e.g., CSTRs) can be concatenated to form a continuous system to achieve conversion of FFA (e.g., complete conversion (i.e., greater than or equal to 99.5 mass percent (mass %) conversion)), separation of pretreated oil (e.g., nearly complete separation (i.e., removal of greater than or equal to 98 mass % of the pretreated oil from the feed stream)), and recycling of alcohol and acid catalyst (e.g., nearly complete recycling (i.e., recycling of greater than or equal to 90 vol % of the initial acid catalyst)). The multiple CSTRs for achieving reaction, separation, and recycling can be arranged in a number of configurations including recycle, parallel processing, and hybrid serial/parallel processing to achieve optimal results for a wide variety of feed stocks having various FFA contents. The process disclosed herein is unique in biodiesel manufacturing at least because other processes do not combine selective esterification chemistry, CSTR series reactor design, adaptive process reconfiguration, and a high efficiency catalyst recycle to pre-treat an oil stream.

The present process can be employed to produce lower alkyl esters (e.g., C₁ to C₄) from an oil source such as natural oil(s) and/or natural fat(s) such as vegetable oils, food grease, soap stock, and animal oils comprising free fatty acids, and combinations comprising at least one of the foregoing oils. Possible oil sources can include, without limitation, vegetable oil, sunflower seed oil, soy bean oil, corn oil, cottonseed oil, almond oil, groundnut oil, palm oil, coconut oil, linseed oil, castor oil, rapeseed oil, industry tallow, abattoir, and combinations comprising at least one of the foregoing. Oils (e.g., waste oil) can be, for example, used cooking oils from restaurants, and the like.

The oil source can be combined with alcohol and an acid catalyst to form the reacting stream. Generally, the alcohol can be a C₁-C₄ alcohol(s), such as methanol, ethanol, isopropanol, butanol, multivalent alcohol(s) (such as trimethylolpropane), as well as combinations comprising at least one of the foregoing alcohols. The selective production of methyl esters without concurrent production of glycerol is achieved using an acid catalyst that has a much higher esterification reaction rate than a transesterification reaction rate. In an exemplary embodiment, the reaction can be carried out with hydrochloric acid (HCl) as the acid catalyst, sulfuric acid, as well as others. In some embodiments of the method disclosed herein, no sulfuric acid is used, wherein in some embodiments, sulfuric acid can be used. In all embodiments, the method can use HCl (or other similar acid catalyst), because the FFA can be selectively converted to methyl esters without the concurrent conversion of the oil to alkyl esters (e.g., methyl esters) and glycerol. This selection occurs in the presence of the alcohol. Likewise, it should be noted that while methyl ester is the exemplary reactant product described herein, the present method is equally suitable for the pretreatment of oil to produce other alkyl esters of fatty acids including, for example, ethyl, propyl, butyl, and so forth.

Turning now to the continuous operation of the process, the selective reaction to pre-treat the oil can be achieved in one or more CSTRs. The number of CSTRs employed will primarily be based on the predicted range of FFA levels in the incoming oil feed streams. Other considerations can include, without limitation, oil quantity and feed rate, process temperatures, process pressures, and the like. It will be appreciated and readily understood by one skilled in the art that the CSTRs disclosed herein can be used in a variety of combinations of size and number to provide the highly selective and continuous pretreatment process. The series of CSTRs enable the system to efficiently handle feed streams with differing amounts of FFA. For example, using an oil feed stream having over 5 mass % FFA, a series of three CSTRs could be used to reduce the FFA level to an acceptably low value (e.g., less than 0.5 mass %). For a feed stream with low FFA levels (e.g., less than 5 mass %), however, only two CSTRs or even a single CSTR could be used to reduce the FFA level to less than 0.5 mass %.

The adaptation of the CSTR system to feed streams with varying levels of FFA can be accomplished by controlling the flow pattern of the incoming feed stream and the methanol and HCl reactive ingredients. In the example where only two of the three CSTRs in a system are required for complete conversion of the FFA, the oil and reactive ingredients feed streams can be rerouted to bypass the first CSTR, thereby reacting in the second CSTR and separating in the third. Alternatively, the feed streams could continue to mix and react in the first CSTR, but the flow pattern can be adjusted to bypass the second CSTR and finish in the third CSTR. The flow pattern of the feed streams throughout the system can be controlled manually (e.g., manual valve control) and/or automatically. In an exemplary embodiment, the adaptive system is achieved automatically by incorporation of sensor and control technology. For example, a sensor can be disposed at an inlet portion of the pretreatment system. The sensor can be configured to determine the FFA level in the oil feed stream. The calculated FFA value can then be sent to a process control computer (i.e., controller) in operative communication with the sensor. The process control computer can be configured to employ control logic and determine the minimum number of CSTRs necessary to reduce the determined amount of FFA in the feed stream to the desired acceptable level (i.e., less than 0.5 mass %). The control computer can be further configured to then send a signal to open and/or close one or more valves in the fluid handling system of the CSTRs in order to adjust the flow pathway of the oil and the alcohol and catalyst in order to use only that minimum number of CSTRs determined by the computer in the pretreatment of the incoming oil. The unused CSTRs in the system can be shut down to reduce power requirements and, thereby, operational costs. In another example, when a new oil feed stream is fed to the system disclosed herein, the above described control exercise of the automated control system can be repeated, and the system can re-adapt to the new FFA level and maintain optimum operation.

The last tank of the CSTR system, regardless of the number of the reactors in the system, can advantageously be configured to operate as a continuous liquid-liquid separator. In one embodiment, the oil stream flows into the last tank (i.e., the separator), already having completely converted the FFA, in a smooth laminar flow. Laminar flow is defined as flow where fluid follows via smooth pathways, does not eddy, and has a Reynold's number, Re, less than or equal to 2,100. This optimal continuous flow allows the alcohol and acid catalyst to separate from the oil and travel to the top of the vessel by virtue of its lower density. The denser pre-treated oil remains in a lower portion of the separator. This separating mechanism works optimally if the inlet flow is smooth and laminar, rather than turbulent and mixed. Moreover, to enable this separating mechanism, an agitator is not necessary in the last tank. The pretreated oil can be continuously removed from the bottom of the separator vessel. In an exemplary embodiment, the pre-treated oil is passed on to a main reactor in operative communication with the CSTR system for reaction of the oil to biodiesel fuel via base catalyzed transesterification, or another like conversion reaction. In a further exemplary embodiment, the alcohol/acid catalyst layer, which has risen to the top portion of the separator vessel, can be recycled to the first CSTR being operated in the system for continued usage in pretreatment of the incoming oil feed stream. In such an embodiment, a lower volume of alcohol and acid catalyst is required to make up the recycle stream and continue to completely convert the FFA than would be necessary without recycle of the reactants.

The process and system disclosed herein is not limited to a particular number of CSTRs in series. The process can apply to any system containing two or more CSTRs, in which the CSTR's may be bypassed and in which the last tank is operated as the separator to facilitate recycle of the alcohol/catalyst stream. For particularly high productivity systems, however, more than two CSTRs would typically be used since a series of CSTRs is more efficient than a single large CSTR capable of handling the high productivity. The process and system can equally apply to a system running two or more CSTR series in parallel. In such a system, multiple CSTR systems connected in series can be run along side one another in parallel, thereby enabling one large system to completely convert multiple streams of oil of varying FFA content simultaneously.

Equation I provides a design basis for the reactor, which illustrates the tradeoff between CSTR volume, throughput, and reaction rate.

$${V}_{CSTR} = V \frac{{[ F F A ]}_{o} - [ F F A ]}{{( d [ F F A ] / d t )}_{{[ F F A ]}_{o}}} \qquad Equation I$$

where:
V_{CSTR}=CSTR reactor volume (liters (l)),
V=volumetric flow entering and leaving the CSTR (in liters per minute l/min)),
[FFA]=concentration of free fatty acid in the mixed inlet feed stream (in grams per liter (g/l)),
[FFA]ₒ=concentration of free fatty acid in the stream exiting the CSTR (in grams per liter (g/l)), and

$$\begin{matrix} where \text{:} \\ {V}_{CSTR} = C S T R reactor volume ( liters ( 1 ) ) , \\ V = volumetric flow entering and leaving the C S T R ( in liters per minute ( 1 \text{/} min ) ) , \\ [ F F A ] = concentration of free fatty acid in the mixed inlet feed stream ( in grams per liter ( g \text{/} l ) ) , \\ {[ F F A ]}_{o} = concentration of free fatty acid in the stream exiting the C S T R ( in grams per liter ( g \text{/} l ) ) , and & \\ {( \frac{d [ F F A ]}{d t} )}_{{[ F F A ]}_{o}} = rate at which free fatty acid reacts in the C S T R at the conditions of the C S T R outlet ( grams per liter \text{-} minute ( g \text{/} l \text{-} min ) ) . & \end{matrix}$$

Equation I can illustrate that two CSTRs in series require less total volume than one CSTR to achieve the same reaction rate and throughput. Each CSTR in a two CSTR system, therefore, can be less than half the volume of a single large CSTR, to achieve the same conversion. Likewise, a series of three CSTRs require even less total volume than the series of two CSTRs, and so on. Economic optimization and production can be used, therefore, to determine the optimal number of CSTRs for a given application. The entire integrated process can be a staged set of CSTR vessels that pre-treat the oil feed stream continuously. As described above, the process is scalable since a wide range of oil streams can be used and production rates achieved by simply selecting the appropriate number of CSTR vessels and appropriate mixed reactant flow path.

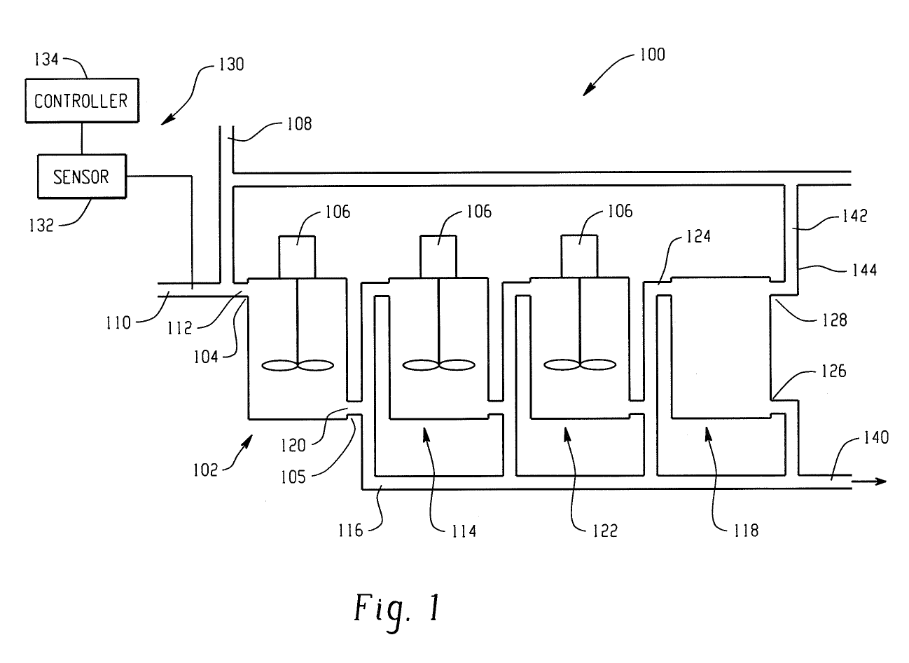

Referring now to FIG. 1, an exemplary embodiment of an oil pretreatment system 100 is illustrated. In this system, four CSTRs are in serial fluid communication. The first CSTR 102 has an inlet 104 and an outlet 105. The inlet 104 can be located in a top portion of the CSTR 102 and the outlet 105 at a bottom portion. In this figure, the alcohol and acid catalyst stream 108 is mixed with the oil feed stream 110 prior to inlet 104 to form a combined feed stream 112. In another embodiment, the first CSTR 102 can have more than one inlet and the one or more of the alcohol, catalyst, and oil can be fed separately and mixed in the CSTR. Agitators 106 are disposed in each of the first, second, and third CSTRs, and are configured to thoroughly mix the oil with the alcohol and catalyst to further encourage the reaction. Although the process can proceed at room temperature, optionally, the oil feed stream 110, alcohol/catalyst stream 108, and/or the combined feed stream 112 can be heated, e.g., to a temperature of about 25° C. to about 75° C., specifically, to of about 50° C. to about 65° C. The combined stream 112 can then be introduced to the first CSTR 102 and the acid-catalyzed esterification reaction can begin. Furthermore, the combined stream can continue to be heated in the CSTR.

The outlet 105 of the first CSTR 102 can be in fluid communication with both the second CSTR 114 and a CSTR system bypass line 116 (which is in fluid communication with the last CSTR, i.e., the separator 118). The first exit stream 120 can, therefore, flow to the second CSTR 114, the separator 118, or can be split between the second CSTR and the bypass line 116. If the FFA content of the oil feed stream 110 is sufficient to require the use of multiple CSTRs, the fluid exiting each of the subsequent CSTRs (e.g., the second CSTR 114 and the third CSTR 122) can also flow to the next CSTR in the series, to the bypass line 116, or be split. In most embodiments, however, the fluid exiting the final CSTR utilized in the series will flow to the fourth vessel—the separator 118; even one or more of the CSTRs (e.g., if the second and/or the third CSTRs) are bypassed. Again, the flow path for the exit stream 120 is dependent upon the FFA content of the oil feed stream 110, and can be determined by, in an exemplary embodiment, an automated system 130 comprising a sensor 132 and controller 134. Each of the first three CSTRs in the series is configured to agitate the combined stream 112 and facilitate complete conversion of the FFA to methyl esters, thereby pretreating the oil.

Again, the last vessel in the system 100 is configured to operate as a separator 118, and therefore, can exclude the agitator 106. Like each of the previous CSTRs, the separator 118 comprises an inlet 124 at a top portion and a first outlet 126 at a bottom portion of the vessel. The separator 118, however, further comprises a second outlet 128 also at a top portion of the vessel opposite the inlet 124. The first outlet 126 is configured to permit continuous exit of a pretreated oil stream 140, which can continue on to a biodiesel conversion system (not shown). Because the oil has a greater density than the alcohol/catalyst liquids, the oil settles to the bottom portion of the separator. The first outlet 126, therefore, can be disposed within the lower about 25% of the separator volume. Likewise, the second outlet 128 is configured to permit a recycle stream 142 to continuously exit the separator and travel via a recycle line 144 back to the alcohol and acid catalyst make-up stream 108.

The lower density of the alcohol and the acid catalyst permit the reactants to rise to the top portion of the separator. For example, in a system using HCl and methanol, the HCl will preferentially partition into the methanol phase due to polarity, and will rise to the top with the lower density methanol. The second outlet 128, therefore, can be disposed within the upper about 25% of the separator volume. In order to attain sufficient residence time and hence separation, the inlet 124 is generally at or above the mid-point of the vessel, or, more specifically, within the upper 25% of the separator, and above the pretreated oil removal point. In order to attain the desired separation of the pretreated oil and reactants (acid catalyst and alcohol), the flow through the separator 118 is preferably laminar and at a controlled velocity that enables the oil to fall to the bottom of the separator as the remaining reactants flow to the top. Moreover, recycling the alcohol and acid catalyst can greatly reduce the need for additional acid catalyst. It was surprisingly discovered, that at least one embodiment of the system was able to reduce FFA content to less than or equal to 0.5 mass % in about 500 gallons of oil with only about 1 gallon of HCl.

Again, the flexibility in fluid flow pathways allows the FFA pretreatment system 100 to be optimized for pretreatment of feed streams containing a wide variety of FFA content. For example, if a feed with low FFA (e.g., less than about 3 mass %) is used, the control system 130 may choose to use only the first CSTR 102, bypass the second and third CSTRs, and to run the fourth CSTR as the liquid/liquid separator 118 to facilitate recycle of the alcohol and catalyst. For a feed stream with a higher FFA content, the system may require the first and second CSTRs to reduce the FFA content to adequate levels, the third CSTR can be bypassed, and the fourth CSTR again used to facilitate separation and recycle. A high FFA content (e.g. greater than about 5 mass %) feed stream may require the first three CSTRs to adequately reduce the FFA, and the fourth CSTR to function as separator and facilitate recycle. Finally, a longer train of CSTRs in series may be used to reduce very high FFA content feeds (e.g., greater than or equal to 25 mass %), and the design of system 100 can permit additional CSTRs to be added to the system at a later time to accommodate such requirements.

As mentioned above, the system 100 can properly pre-treat the oil feed stream such that it is ready for full conversion into biodiesel fuel. A biodiesel conversion system utilizing base catalyzed transesterification, or the like, therefore, can be in operative communication with the system 100. The biodiesel system can be configured to produce ASTM quality biodiesel fuel from the pre-treated oil stream 140. ASTM quality biodiesel requires greater than 99 mass % conversion and greater than 99.8 mass % glycerol removal. Although some other systems may claim ASTM quality processing, the National Biodiesel Board estimates that, in 2006, more than half of all biodiesel produced in the USA was not ASTM grade.

Recoverability and reusability of the catalyst employed can also be determined using the above described method. The concentration of acid in the aqueous layer can be calculated. For example, the concentration of sulfuric acid in the aqueous layer can be calculated using an equation provided from a study conducted by Evans et al. on the electrolytic dissociation of sulfuric acid in aqueous methanol. The dissociation of sulfuric acid in water (R1-R2) is shown below.

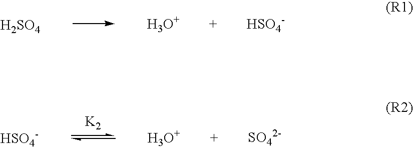

There is a strong effect on the dissociation constant of sulfuric acid in 10 volume percent (vol %) to 20 vol % aqueous methanol. The equation used to calculate the second dissociation constant (K₂) is shown below.

pK₂=1.669+0.0336 m+0.0126 T(° C.) Equation II

In Equation II, m is the vol % of methanol in the aqueous solution and T is the temperature of the solution during titration. It can be assumed that the first dissociation for sulfuric acid is in effect infinity and goes to completion. The resulting mole balance for the titration given by shown in Equation III.

n_{OH}_{^{−}}_{,added}=(2n_{SO}_{₄}_{^{2−}}+n_{HSO}_{₄}_{^{−}})_{in solution} Equation III

In Equation III, n represents number of moles. The equilibrium equation for the second dissociation is shown in Equation IV.

$${K}_{2} = \frac{[ {H}_{3} {O}^{+} ] [ {SO}_{4}^{2 -} ]}{[ {HSO}_{4}^{-} ]} \qquad Equation IV$$

Between Equations II and III there are two unknowns, [HSO₄⁻—] and [SO₄²⁻] in the sample, which can be solved for using the system of two equations.

The effect of water, accumulated in the methanol phase during esterification, on the reuse of the catalyst and methanol can be determined by comparing the observed rate constant between runs. The rate expression employed can be a second order, elementary, reversible relationship shown in Equation V.

r_{FFA}⁻={k_{FFA}[A]}[FFA][MeOH]−{k_{FFA}−[A]}[FAME][H₂O] Equation V

In Equation V, [A] is the concentration of the acid catalyst; because the catalyst is not consumed during the reaction, [A] is a constant and can be combined with the rate constant k_{FFA}. Equation V can be written in terms of FFA conversion, where the equation for conversion is given below.

$$x = \frac{{[ FFA ]}_{o} - [ FFA ]}{{[ FFA ]}_{o}} \qquad Equation VI$$

where:
x=FFA conversion
[FFA]=final concentration of free fatty acid (in mol per liter (mol/l)),
[FFA]ₒ=initial concentration of free fatty acid (in mol per liter (mol/l))

Combining equations V and VI, the rate expression can be written in terms of FFA conversion. Due to a large molar excess of methanol, the concentration of methanol is assumed to be constant at its initial concentration. Reverse hydrolysis can be neglected, yielding the rate expression below.

$$\frac{d x}{d t} = \{ {k}_{FFA} [ A ] \} ( 1 - x ) ( - x ) \qquad Equation VII$$

In Equation VII, [symbol] is the ratio of the initial methanol concentration to the initial FFA concentration, ([MeOH]ₒ/[FFA]ₒ). By integrating Equation VI from t=0 to t and substituting k_{FFA}[A]=k₁, the following expression can be used to linearize initial FFA concentration to yield a rate constant from the slope.

$$ln ( \frac{- x}{1 - x} ) - ln ( ) = {{k}_{1} [ FFA ]}_{o} t \qquad Equation VIII$$

In biodiesel production, it is desirable to limit waste streams and recover reactants. After acid catalyzed esterification of free fatty acids in the presence of triglycerides, methanol can be recovered by phase separation. The reuse of catalyst can be limited by the formation of a glycerol layer and its tolerance to accumulating water produced by esterification in the methanol layer.

The recovered catalyst can travel through the recycle line 144 back to the alcohol and acid catalyst make-up stream 108. According to the embodiment illustrated in FIG. 1, the recovered catalyst can then be mixed with the oil feed stream 110 prior to the inlet 104 to form a combined feed stream 112. In another embodiment, the first CSTR 102 can have more than one inlet and the one or more of the alcohol, recycled catalyst, and oil can be fed separately and mixed once in the CSTR.

The following examples are merely exemplary and are intended to further explain and not to limit the process and system disclosed herein.

### EXAMPLES

The basic process configuration with a single continuous stirred tank reactor/separator vessel was realized for Examples 1 and 2. The reactions were run using several types of vegetable oil combined with methanol and a hydrochloric acid (HCl) catalyst. The reaction was completed to estimate the chemical kinetics of the FFA reaction with the HCl catalyst.

### Example 1

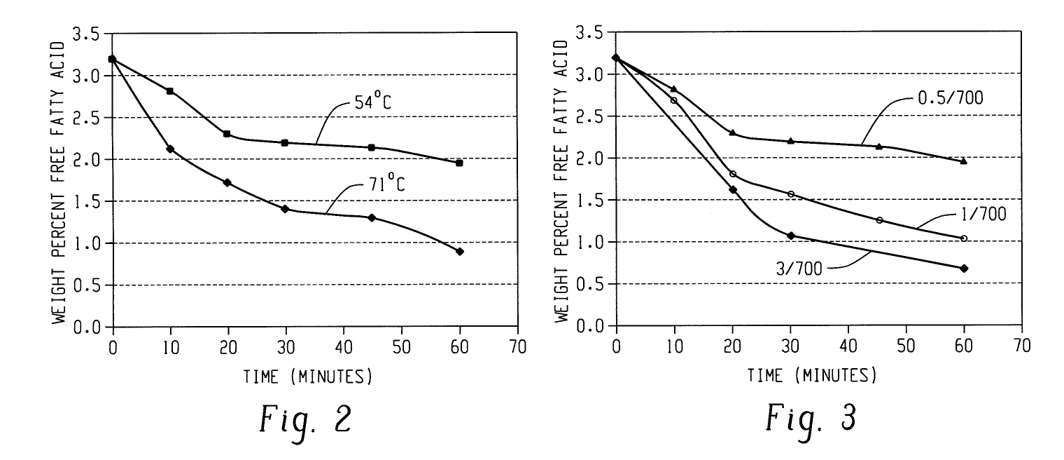

A 1,000 milliliter (mL) flask served as the CSTR/separator in this example. Three different oils were tested. In each sample, 700 mL of the oil was mixed with 200 mL of methanol and various volumes of HCl in the 1,000 mL flask on a magnetic stirrer at temperatures of about 54° C. to about 71° C. Sample 1 included 700 mL of a soy oil containing about 3 mass % FFA. Sample 2 included 700 mL of a canola oil containing about 1.2 mass % FFA. And Sample 3 included 700 mL of a commercial yellow grease containing about 3.2 mass % FFA. Results from the reaction with the yellow grease are illustrated in FIG. 2. FIG. 2 shows that the reaction proceeds more quickly at higher temperatures (about 71° C.), and that the conversion of FFA goes through a plateau period from about 20 to about 40 minutes into the reaction. FIG. 3 shows the FFA conversion as a function of time for varying HCl concentrations, with the concentrations in units of mL acid per 700 mL of oil (e.g., 0.5/700 is 0.5 mL HCl per 700 mL of oil). As can be seen, FIG. 3 further indicates that increasing the acid catalyst (HCl) concentration also increases the speed of the FFA conversion.

### Example 2

In the second experiment, larger scale reactions were conducted in a 100 gallon CSTR/separator vessel having a valved outlet at the bottom of the vessel. The reaction kinetics were verified under less ideal mixing conditions (e.g., although an agitator was used, thorough mixing was not attained). For Example 2, only commercial yellow grease having a FFA concentration of 3.2 mass % was used. The concept of separating and recycling was able to be tested and verified in the larger vessel. Again, the grease was reacted with methanol and HCl according to the amounts reproduced in Table 1 below.

TABLE 1

| | | Oil | FFA start | CH₃OH | HCl | | | FFA end |
|---|---|---|---|---|---|---|---|---|
| Sample | | Vol. | conc. | Vol. | Vol. | Temp. | Reaction | (mass |
| No. | Experiment | (gal.) | (mass %) | (gal.) | (gal.) | (° C.) | time (hr) | %) |
| 4 | Yellow | 500 | 3.2 | 20 | 1 | 22 | 14 | 0.75 |
| | Grease | | | | | | | |
| 5 | waste grease 1 | 30 | 6.5 | 7.5 | 0.0879 | 22 | 16.5 | 0.86 |
| 6 | waste grease 2 | 50 | 5.5 | 12.5 | 0.1849 | 22 | 14 | 0.8 |
| 7 | waste grease 3 | 25 | 6 | 5.5 | 0.066 | 22 | 14.5 | 0.78 |

Based upon the above examples, it can be seen that the present system utilizing the selective HCl catalyst in the methanol reactant can reduce the FFA content in a oil to suitable levels, thereby permitting the use of the pre-treated oil in a base catalyzed transesterification biodiesel conversion reaction.

The following examples compare sulfuric acid (H₂SO₄) and hydrochloric acid (HCl) as catalysts for the esterification of linoleic acid with methanol in the presence of soybean oil triglycerides. Catalyst recoverability after esterification was investigated at several different initial concentrations of linoleic acid in soybean oil. The effect of water, as a product of esterification, was studied by reusing the recovered methanol layer for esterification of linoleic acid in the presence of soybean oil. The effect of accumulating water in the methanol layer was also studied by repeating experiments and changing initial water concentrations.

### Example 3

In the following Samples, temperature was held constant at 70° C. for all Samples tested. Reactions were carried out in a 1 Liter (L) three-neck round bottom flask with a water-cooled reflux condenser to minimize methanol losses. The flask was submerged in a water bath placed on a temperature controlled magnetic stirring hotplate from Fisher Scientific. The reaction temperature was monitored by thermocouple and a mercury thermometer and held within plus or minus 1° C. of 70° C. Agitation was provided by a magnetic stirrer (1.5 in.×0.75 in. diameter/3.81 cm×1.91 cm) at 700 revolutions per minute (rpm). Sample 12 was repeated at 800 rpm and the initial rate of disappearance of linoleic acid was compared to the same experiment conducted at 700 rpm to verify that the mixing was sufficient to prevent bulk mass transfer limitations.

Linoleic acid concentrations ranged from 2 mass percent (mass %) to 15 mass % of the initial soybean oil-fatty acid mixture. Acid catalyst concentration was held constant at 1 mass % of soybean oil triglycerides and initial methanol concentration was approximately 6:1 molar ratio with respect to soybean triglycerides. Table 2 provides the six formulations used in Example 3, where 400 grams (g) of soybean oil were used in each case.

TABLE 2
Initial Conditions of Experimental Formulations

| Sample | | Volume of Acid | Weight Linoleic Acid |
|---|---|---|---|
| No. | Acid Catalyst* | Solution (mL) | (g) |
| 8 | H₂SO₄ (97 vol %) | 2.25 | 8.20 |
| 9 | H₂SO₄ (97 vol %) | 2.25 | 21.0 |
| 10 | H₂SO₄ (97 vol %) | 2.25 | 70.8 |
| 11 | HCl (36 vol %) | 9.15 | 8.20 |
| 12 | HCl (36 vol %) | 9.15 | 21.0 |
| 13 | HCl (36 vol %) | 9.15 | 70.8 |

*acid catalyst is in aqueous solution in the volume percent set forth.

Virgin soybean oil (400 g of Whole Harvest 100% Soy Product) was loaded into the reaction flask and heated to 70° C. The desired amount of linoleic acid (Acros Organics, 60 mass %, Tech grade (32 mass % Oleic acid, 8 mass % saturated carbon to 18 mass % fatty acid)) was measured separately and added to the reaction flask. Methanol (111 mL±0.5 mL of 99.9% high performance liquid chromatography (HPLC) grade, Fischer Scientific) was measured into a separate 250 mL flask. The amount of added methanol was calculated to correspond exactly to a 6:1 molar ratio of methanol:triglyceride (TG) for the case of pure triolein (weight average molecular weight (Mw) equal to 885 grams per mol (g/mol)). However, since the weight average molecular weight of Soybean TG is typically reported in the range of 860 to 880 g/mol, the actual molar ratio employed in Samples 8-13 is more accurately 5.95:1. The methanol:linoleic acid molar ratios were 93.7, 36.6, and 10.86 for FFA levels of 2 mass %, 5 mass %, and 15 mass %, respectively. Concentrated acid catalyst solution was added to the 250 mL flask using a graduated pipette and mixed with the methanol. Both reagent grade hydrochloric acid (12 Molar—36%) and reagent grade sulfuric acid (18 Molar—97%) were used as catalysts. The acid solution volume was adjusted to add acid at 1 mass % of the soybean TG, specifically 4 g±0.1 g.

For each of Samples 8-13, ten milliliter (mL) samples were withdrawn periodically and immediately quenched in an ice bath for 5 minutes. These 10 mL samples were then centrifuged (using a machine from Thermo Electron Corp., model HN SII) at 4000 revolutions per minute (rpm) for 2 minutes. After centrifugation, the methanol layer was removed from each sample in a 250 mL separatory funnel and discarded. To remove any residual methanol and acid, the oil phase was washed with distilled water in a 250 mL separatory funnel by adding 50 mL of distilled (DI) water, shaking vigorously, and allowing to settle for 5 minutes. After settling, the water layer was discarded and each sample was centrifuged again for 2 minutes at 4000 rpm to remove residual water.

The withdrawn samples were prepared for gas chromatography following ASTM 6584-00 for analysis of free and total glycerine content in biodiesel. The derivatized solution was injected (e.g., in an amount of 1 microliter (μL)) into a Hewlett-Packard 5890 Series II Gas Chromatograph equipped with a Quadrex Aluminum Clad column with a 1 meter retention gap and employing a flame ionization detector to determine fatty acid methyl-ester, glycerol and glyceride (tri-, di-, mono-) concentrations. Computer-assisted analysis of the resulting chromatograms was performed using Chem-Station software (Hewlett-Packard, now Agilent Technologies). Samples were also prepared for titration following ASTM D664-00 for acid number of biodiesel samples. After allowing the diluted sample to mix for one minute, titrant was added drop by drop until the permanent light pink endpoint was reached.

Samples 14-19 were repeated to study the recoverability of the unreacted methanol and acid catalyst. Recoverability experiments were conducted as described above with respect to Samples 8-13 (i.e., Sample 14 corresponds to Sample 8, Sample 15 corresponds to Sample 9, etc.), but no samples were withdrawn. The reactants were mixed at 70° C. for the time period required for the concentration of linoleic acid to reach approximately 0.2 mass %, with the time estimated from the results of Samples 8-13. After stopping agitation, the reaction flask was placed in an ice bath where the phases were allowed to separate for 2 hours. The oil phase was decanted using a 1 L separatory funnel. A sample of the oil phase was washed and prepared for titration as previously described.

The recovered methanol layer was decanted into a 100 mL graduated cylinder to measure its volume. The recovered methanol phase was prepared for titration as 10% (by volume) aqueous methanol solutions, where 10 mL of the recovered methanol layer was added to 90 mL of distilled water in a 100 mL graduated cylinder. Titrant was made by dissolving 44 grams of potassium hydroxide pellets (87.9%, JT Baker) in 392 mL of distilled water to make a 1.74 M KOH solution. Titrant was added drop by drop until the phenolphthalein endpoint was reached.

For the initial condition of 5 mass % linoleic acid (Samples 9, 15 and 12, 18), the recovered methanol layer containing the recovered acid catalyst and water, was reused with a new mixture of 5 mass % linoleic acid in 400 g of soybean oil triglycerides. Due to loss of methanol by consumption, partitioning, and small amounts of evaporation, a small amount of fresh methanol was added to match the 111 mL of methanol initially used. No water was removed from the recovered methanol. Samples were withdrawn from Samples 9 and 12 periodically and treated as previously described. These experiments illustrate that substantially all the acid (>95%) is recovered in the methanol layer after phase separation.

To investigate multiple reuse of the methanol and acid where further accumulation of water in the methanol layer is expected, Samples 20 and 21 were conducted repeating the conditions of Samples 9 and 12 as previously described adding different concentrations of distilled water to the methanol and acid before the reaction. Samples were withdrawn from Samples 9 and 12 periodically and treated as previously described. These experiments illustrate that acid can be recovered over and over, and also illustrates that hydrochloric acid maintains its catalytic activity much better than sulfuric acid as water concentration builds up.

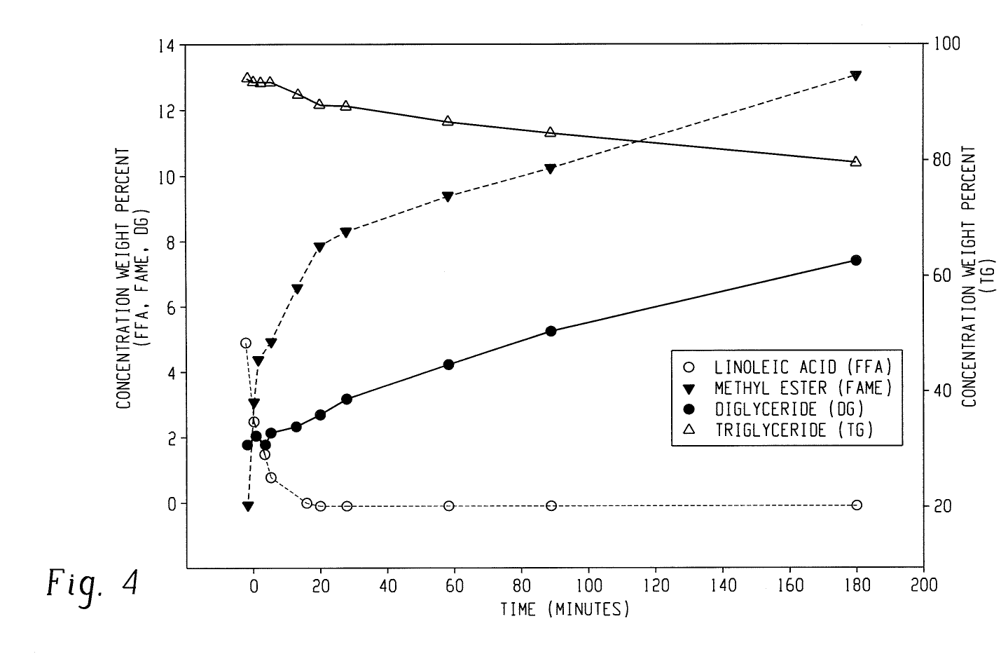

For Samples 8-13, samples were also monitored for FFA, bound glycerine (MG, DG, TG), and free glycerine (G). FIG. 4 shows a typical data set, specifically from Sample 9 at the initial condition of 5 mass % initial linoleic acid with sulfuric acid catalyst. FIG. 4 illustrates that the free fatty acid and triglyceride concentrations decrease with time and the fatty acid methyl ester and diglyceride concentrations increase over time.

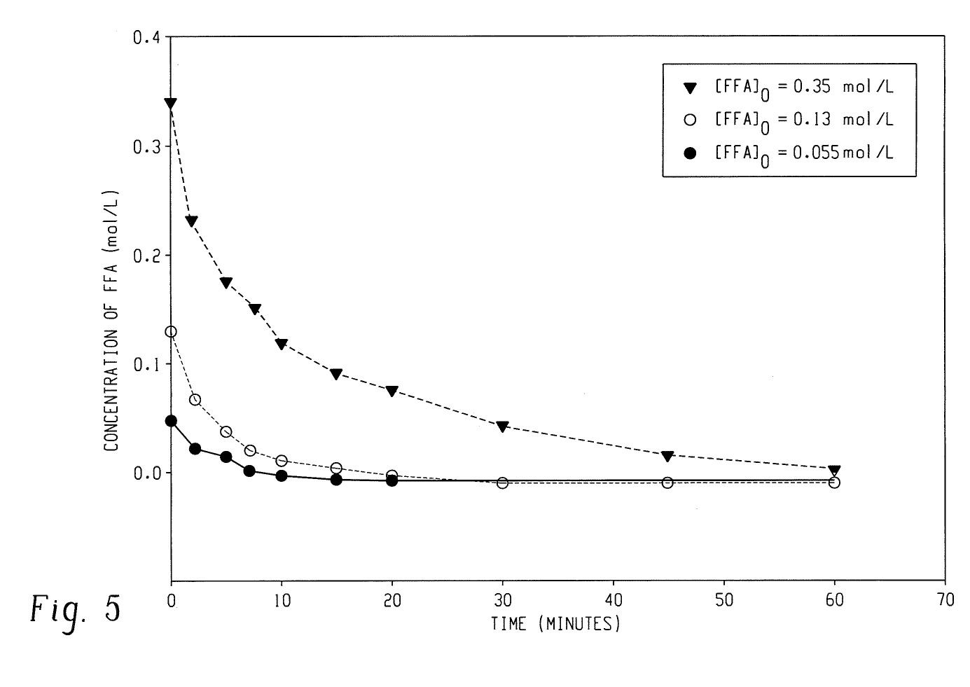

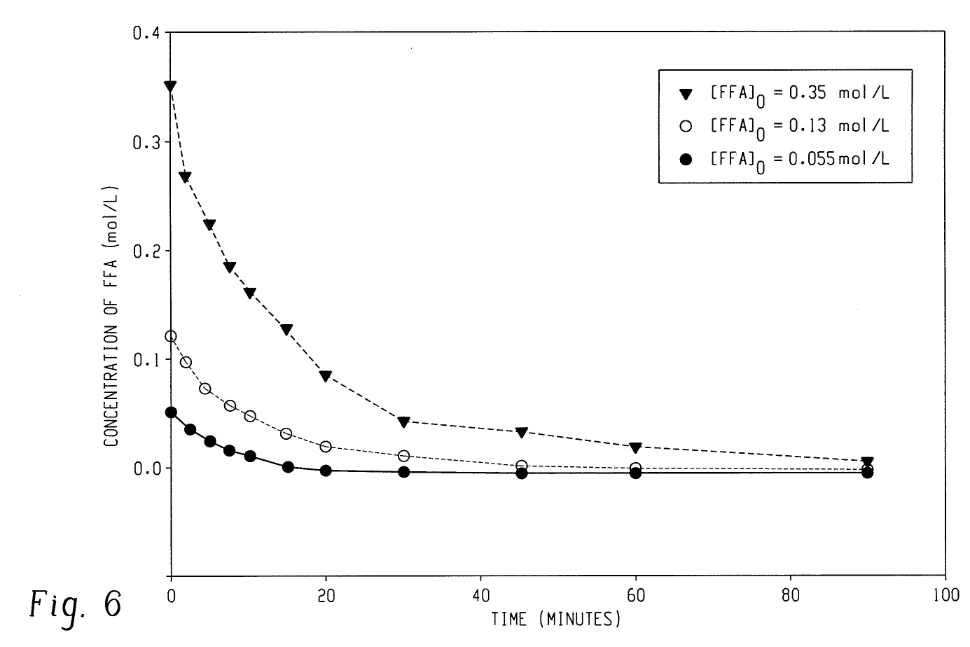

Reproducibility was verified by repeating several of the above described experiments. The measured values of the chemical composition remained within 5% of initial measurements in all cases. As shown in FIG. 4, there was significant conversion of FFA to methyl ester. Negligible amounts of monoglyceride and glycerol were present, so they do not appear in FIG. 4. Transesterification of soybean triglycerides did not nearly reach completion; however, there was notable conversion of triglycerides to diglycerides and methyl ester. The majority of transesterification took place after esterification already had reached equilibrium. It was noted that conversion of TG to DG was less than 5 mass % for all of Samples 8-13 before the concentration of FFA reached 0.2 mass %. FIG. 5 shows the concentration of linoleic acid with time, using sulfuric acid as the catalyst, at different initial concentrations of linoleic acid. FIG. 6 displays an equivalent plot using hydrochloric acid as a catalyst.

Recoverability of each acid catalyst was determined by repeating Samples 8-13, but withdrawing no samples. Experiments were allowed to run until the concentration of linoleic acid reached 0.2 mass % for Samples 8, 9, 11, and 12, and 0.5 mass % for Samples 10 and 13. After rapidly cooling the final reaction mixture, it was allowed to separate for two hours, after which each phase was titrated as previously described. After settling, the observed phases were a methanol rich phase containing water and an acid catalyst, and an oil rich phase. Slight differences were observed after decanting; a lighter yellow color was observed after esterification of 15 mass % FFA in soybean oil with sulfuric acid, and a darker orange after using hydrochloric acid. The dark orange color was not observed when initial FFA concentration was 5 mass % or less.

Table 3 summarizes the results from the recoverability experiments. Table 3 demonstrates that Samples 11-13 utilizing hydrochloric acid as the catalyst, have a greater volume of methanol layer recovered and more grams of catalyst recovered. For instance, comparing Sample 9, with 3.82 g to 3.90 g of catalyst recovered and Sample 12, with 3.95 g to 3.99 g of catalyst recovered shows that up to 1.3% more catalyst was recovered using hydrochloric acid (Sample 12) versus sulfuric acid (Sample 9). The volume of the methanol layer recovered was 10 mL or 12% greater using hydrochloric acid versus sulfuric acid when comparing Samples 9 and 12. Comparing Samples 8 and 11, it can be seen that Sample 11, using hydrochloric acid had a greater volume of methanol layer recovered by 11 mL or 12%. A comparison of Samples 10 and 13 shows an even greater difference between the volume of methanol layer recovered when using hydrochloric acid versus sulfuric acid catalysts. There is a 15 mL or 19% difference in the volume of the methanol layer recovered when hydrochloric acid (Sample 13) was used versus sulfuric acid (Sample 10). In other words, when using hydrochloric acid (e.g., when the catalyst comprises greater than or equal to 80 vol % hydrochloric acid, specifically, greater than or equal to 90 vol %), greater than or equal to a 5% difference (i.e., more methanol recovery when using hydrochloric acid versus sulfuric acid), specifically, greater than or equal to 8%, and more specifically, greater than or equal to 10%, and even greater than or equal to 15% possible, when compared to the same system using sulfuric acid instead of the hydrochloric acid. These results indicate that hydrochloric acid is not only a suitable catalyst for use in the method described herein, as more methanol can be recovered and reused in the process; hydrochloric acid has unexpectedly superior results compared to sulfuric acid. These results further illustrate that in a continuous process with recycle, hydrochloric acid should perform better and for a longer period of time than sulfuric acid. This is an unexpected result because the industry has converged on the use of sulfuric acid in most processes because the sulfuric acid performs better under very dry conditions. This example takes into account the difference between pristine laboratory conditions and realistic industrial conditions where more water will be present.

TABLE 3
Summary of Recoverability Experiment Results

| | | | | | | Volume of |
|---|---|---|---|---|---|---|
| | | Initial | | | Catalyst | Methanol Layer |
| | | FFA | Rxn | mass % | Recovered | Recovered |
| No. | Catalyst* | (mol/L) | Time (min) | FFA Final | (g) | (mL) |
| 8 | H₂SO₄ | 0.055 | 10 | 0.18 | 3.92 | 82 |
| | (97%) | | | | | |
| 9 | H₂SO₄ | 0.13 | 20 | 0.19 ± 0.05 | 3.86 ± 0.04 | 74 ± 1 |
| | (97%)** | | | | | |
| 10 | H₂SO₄ | 0.35 | 60 | 0.48 | 3.75 | 66 |
| | (97%) | | | | | |
| 11 | HCl (36%) | 0.055 | 20 | 0.19 | 3.96 | 93 |
| 12 | HCl | 0.13 | 60 | 0.23 ± 0.04 | 3.97 ± 0.02 | 84 ± 1 |
| | (36%)** | | | | | |
| 13 | HCl (36%) | 0.35 | 90 | 0.53 | 3.86 | 81 |

*acid catalyst is in aqueous solution in the volume percent set forth.
**Results are an average of three repeated experiments.

In order to examine reusability of each catalyst, Samples 9 and 12 were repeated and the methanol layer was recovered after 2 hours of decanting. Methanol was added to the aqueous layer to make up for methanol consumed by reaction and methanol lost by partitioning in the oil phase and small amounts of evaporation loss. It was assumed that all water produced by esterification ended up in the methanol layer. The methanol/acid/water mixture was then reused to treat the same amount of linoleic acid as the first run (5 mass % linoleic acid in 400 g Soybean Oil). To investigate further accumulation of water in the methanol layer, Samples 9 and 12 were repeated adding different concentrations of distilled water to the methanol and acid before the reaction. Samples were withdrawn periodically and treated as previously described.

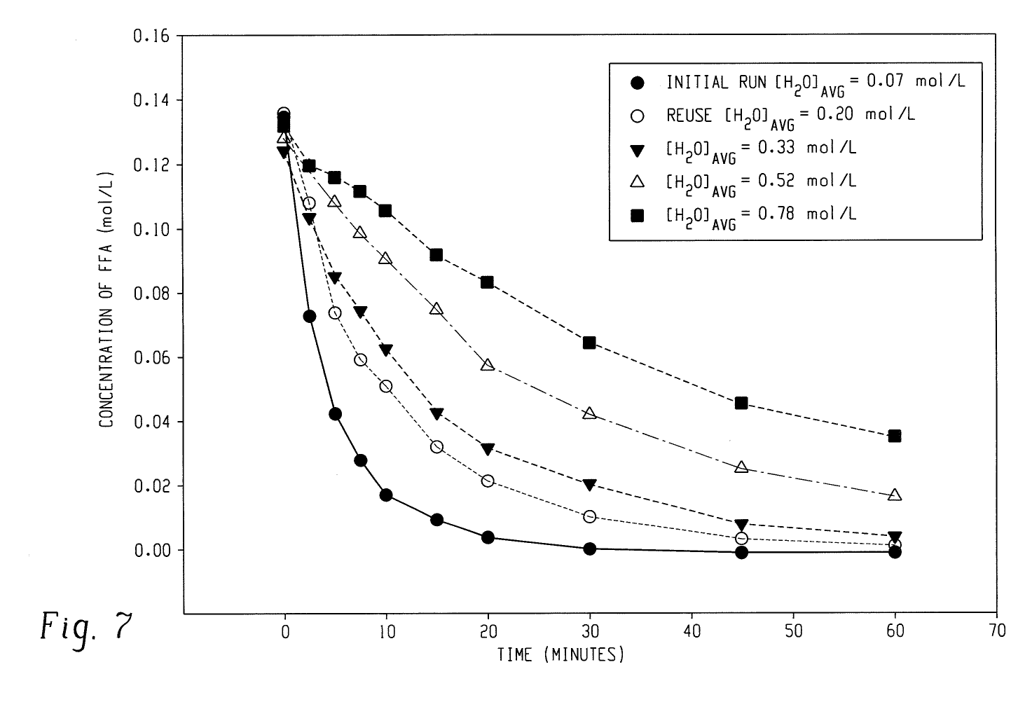

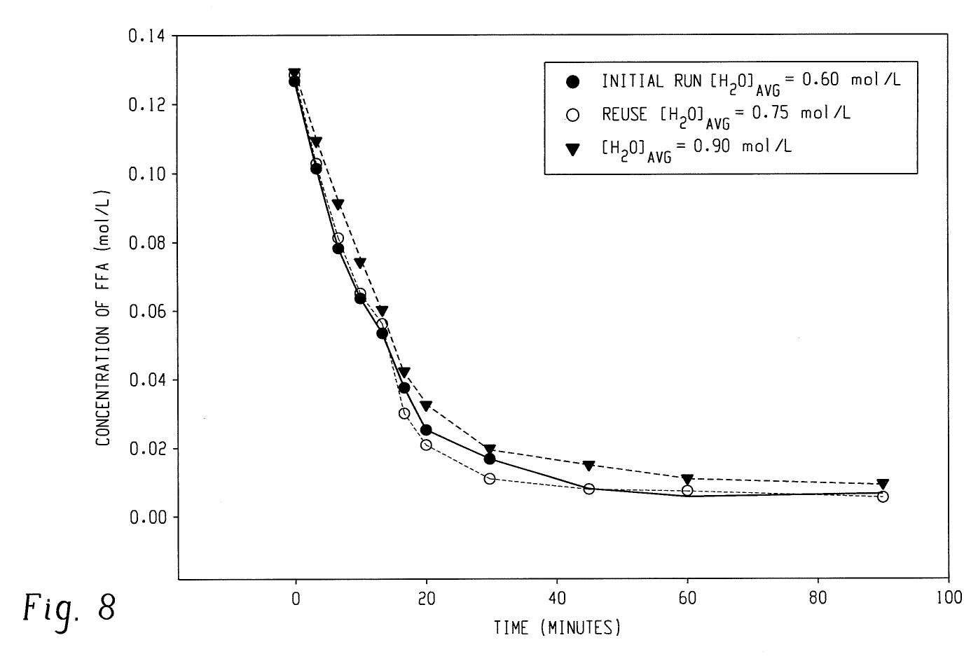

FIG. 7 displays the concentration of linoleic acid with time for esterification catalyzed by sulfuric acid for the initial run compared to its reuse and experiments with increased initial water concentration. FIG. 8 is a similar plot for experiments where hydrochloric acid was used for the catalyst. Because water was being continuously produced by esterification, water concentrations are denoted as average water concentration throughout the experiment. In FIGS. 7 and 8, the free fatty acid concentration versus time is illustrated where the initial free fatty acid concentration is equal to 0.13 mol/L for various water concentrations. As can be seen by FIGS. 7 and 8, lower or similar amounts of free fatty acid can be achieved at higher water concentrations using hydrochloric acid (FIG. 8) versus sulfuric acid (FIG. 7), indicating that water does not have as great an effect on the reusability of the catalyst in systems utilizing hydrochloric acid as the catalyst. For example, consider the last set of points in each of FIGS. 7 and 8. In FIG. 7 (using sulfuric acid), beginning water concentration was 0.78 mol/L, while in FIG. 8 (using hydrochloric acid), beginning water concentration was 0.90 mol/L. In FIG. 8, the free fatty acid content decreased to 0.02 mol/L in approximately 30 minutes, but as shown by FIG. 7, the free fatty acid content did not decrease below 0.05 mol/L, even after 60 minutes when sulfuric acid was used.

As can be seen from both FIGS. 7 and 8, the concentration of free fatty acid decreased with time using both catalysts. However, as shown in FIG. 8, when using hydrochloric acid, the concentration of free fatty acid decreased more quickly and plateaued in less time versus the samples using sulfuric acid for the initial run, reuse, and experiments with greater initial water concentrations.

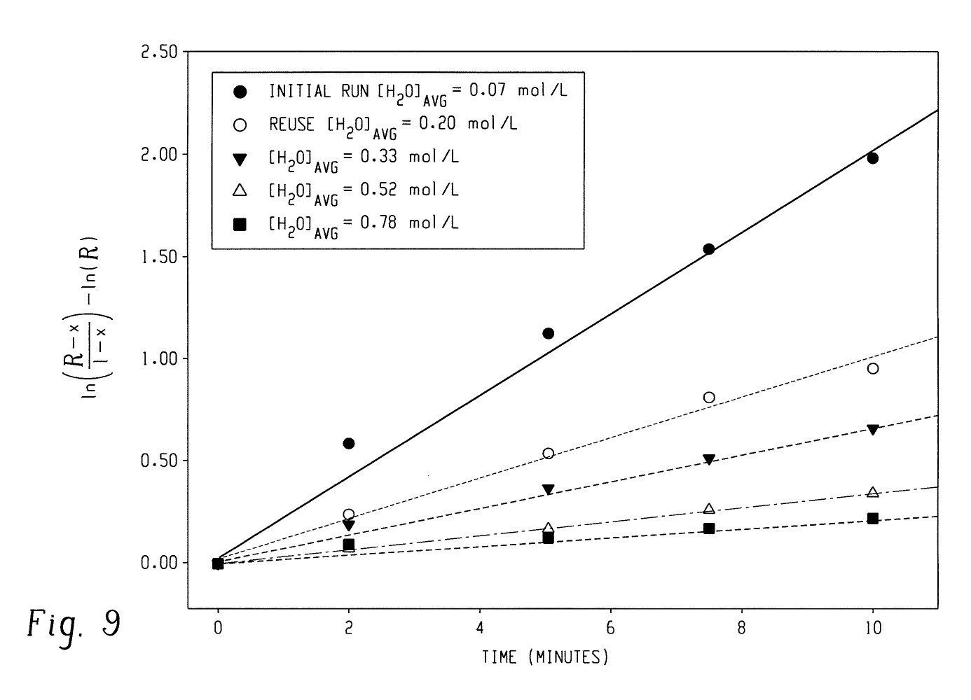

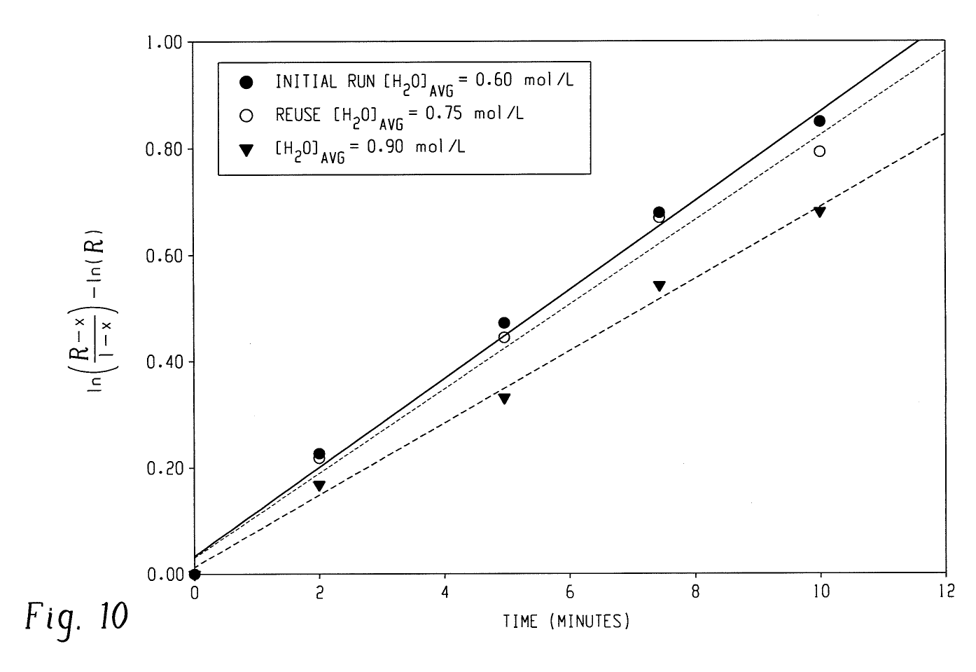

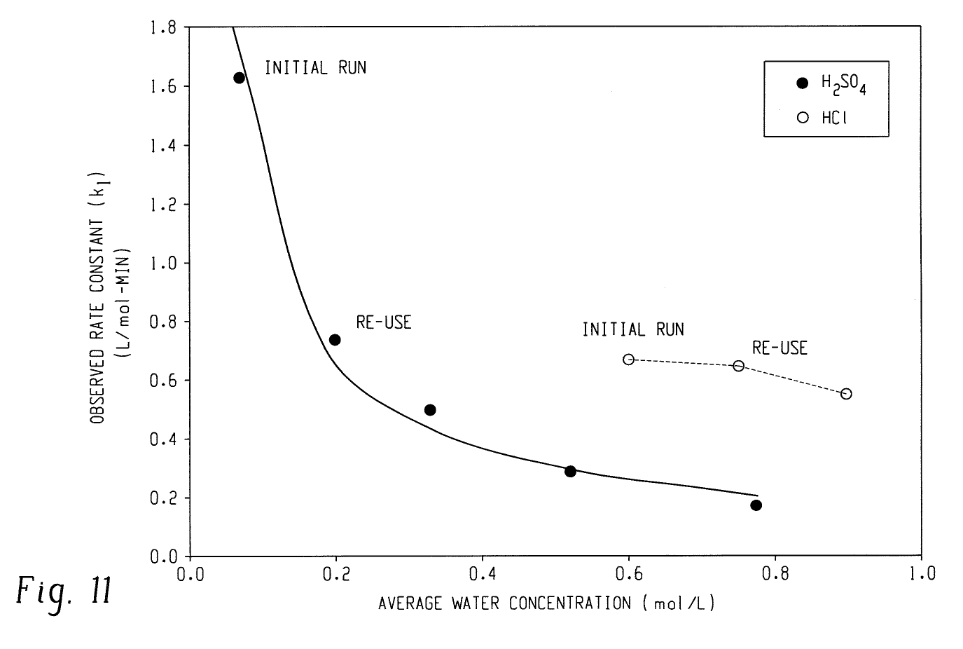

FIG. 9 displays the linearization of the initial FFA conversion data when sulfuric acid was used for a catalyst and the initial concentration of FFA was 5 mass %; individual data sets are at different initial water concentrations. FIG. 10 shows the equivalent plot where hydrochloric acid was used as the catalyst. In both FIGS. 9 and 10, the initial concentration of free fatty acid was 0.13 mol/L for various water concentrations. Using best fit slopes from FIGS. 9 and 10, a rate constant was derived, and the effect of average water concentration on the observed rate constant was examined. FIG. 11 shows the effect of water concentration on the observed rate constant for both catalysts at 70° C. Because hydrochloric acid was purchased as a 36 vol % aqueous solution, the first data point is at a relatively high water concentration of 0.60 mol/L.

The experiments above were conducted with hydrochloric acid and sulfuric acid levels that range from 11 mass % to 50 mass % with respect to the FFA. Those levels, which correspond to 1 mass % with respect to the soybean triglycerides, are quite high for homogeneous catalysis of FFA. Note, however, that the chemical system of reactions is quite complex as the hydrochloric acid and sulfuric acid slowly catalyze the transesterification of the triglycerides as well as more rapidly catalyze the esterification of the free fatty acids.

Several additional experiments were therefore conducted at lower hydrochloric acid and sulfuric acid levels. Samples 9 and 12 from Table 3 contained 5 mass % FFA relative to triglycerides and 20 mass % acid catalyst relative to the FFA. Additional experiments were conducted with 5 mass % FFA and with acid catalyst levels of 2, 4, and 10 mass % with respect to FFA to verify that the results depicted above for FFA are not simply due to an overabundance of acid catalyst. Various amounts of water were added to the reaction mixtures for comparison with the results above.

Just as above, hydrochloric acid activity was less sensitive to the water than sulfuric acid activity, but the formulations using low levels of catalyst converted FFA too slowly for industrial needs. For example, the mixture containing 2 mass % sulfuric acid (with respect to the FFA) and roughly 0.5 mass % water reduced the FFA from 5 mass % to 1.8 mass % after 90 minutes, whereas at 20 mass %, sulfuric acid (with respect to the FFA), the FFA was reduced from 5 mass % to 1.8 mass % in less than 5 minutes. The mixture containing 2 mass % hydrochloric acid (with respect to the FFA) and 0.5 mass % water reduced the FFA from 5 mass % to 1.8 mass % in roughly 30 minutes and to 0.3 mass % in 90 minutes, about ⅓ the conversion rate of the case with 20 mass % hydrochloric acid (with respect to the FFA). These results further demonstrate that hydrochloric acid is less sensitive to water than sulfuric acid.

Another comparison between hydrochloric acid and sulfuric acid was conducted where the number of moles of hydrochloric acid and sulfuric acid were matched. At 4 mass % hydrochloric acid and 10 mass % sulfuric acid, both with respect to FFA, the number of moles is the same due to differences in molecular weight. Using a water content of approximately 2 mass %, the experiment with hydrochloric acid reduced the FFA content from 5 mass % to roughly 2.2 mass % in 30 minutes, while the experiment with sulfuric acid reduced the FFA content from 5 mass % to roughly 2.9 mass % in 30 minutes. Thus, if significant levels of water reside in the mixture, hydrochloric acid may be more effective than sulfuric acid, and high levels of acid may be used. In other words, where there is greater than or equal to 1 mass % water, specifically, greater than or equal to 2 mass % water, and more specifically, greater than or equal to 3 mass % water, with respect to the triglycerides, hydrochloric acid is more effective than sulfuric acid (when catalyzing using the same number of moles of each acid).

In yet another example, the sulfuric acid treatment methodology was attempted in an industrial setting on a commercial yellow grease of roughly 9 mass % FFA. The FFA could only be reduced to 3 mass % without costly decanting and a complete second stage using new acid catalyst and methanol. However, with the above disclosed hydrochloric acid process, the FFA reduction was from roughly 9 mass % to a level of 0.5 mass % in a volume of 1,800 gallons of yellow grease (i.e., without decanting and without a second stage using new acid catalyst and methanol).

Increasing interest has been invested in renewable, environmentally benign alternative fuels due to greenhouse gas emissions and volatile oil prices. Biodiesel is renewable, and an immediate replacement for petroleum diesel. A key challenge in economically efficient biodiesel production is the utilization of waste cooking oils and animal fats with high concentrations of free fatty acids. Homogenous acid catalysts have been employed for the esterification of FFA for treatment of waste oils, however in many cases they are not recovered and reused.

Within the range of conditions tested, it was determined that transesterification is negligible when either sulfuric acid or hydrochloric acid are used to catalyze the esterification of polyunsaturated 18:2, 18:3 linoleic acid in the presence of soybean oil triglycerides. Greater than 95 mass % of the catalyst was recovered in the methanol layer under all conditions investigated.

The system and process described herein advantageously illustrates the potential for recoverability and reusability of acid catalysts, specifically hydrochloric acid and sulfuric acid catalysts for efficient pre-treatment of waste cooking oils for subsequent conversion to biodiesels. For example, esterification of omega-9 polyunsaturated fatty acids, specifically 18:2, 18:3 linoleic acid with methanol and a homogenous acid catalyst was investigated over a range of fatty acid concentrations and determined to have greater than 95 mass % recoverability after esterification under all test conditions. It was also discovered that when recovered methanol was used containing recovered catalyst and water, hydrochloric acid catalyzed esterification exhibits a higher tolerance to water accumulation than recovered sulfuric acid. For example, after sulfuric acid was recovered and reused, the observed rate constant decreased more than 50% to a value comparable to that observed for hydrochloric acid at more than three times the water concentration. Thus, hydrochloric acid demonstrates higher tolerance for accumulating water in the methanol layer.

The system and process described herein advantageously combines selective transesterification chemistry, CSTR series reactor design, adaptive process reconfiguration, and high efficiency catalyst recycle to produce a pre-treated oil for conversion to biodiesel from a commercial natural oil. The system and process is uniquely configured to handle highly variable material feed streams. Moreover, the process utilizes a high selectivity reaction in which acid catalyzed esterification converts FFA to alkyl esters without converting any of the oil to methyl esters and glycerol. For example, FFA conversions to less than or equal to 0.5 mass %, specifically, less than or equal to 0.3 mass %, and more specifically, less than or equal to 0.2 mass %, can be attained with the present process and system. Finally, the present system and process incorporates all of the above advantages while in continuous operation, and wherein the catalyst and one reactant (alcohol) is recycled and reused to further reduce operating costs.

Ranges disclosed herein are inclusive and combinable (e.g., ranges of “up to about 25 mass %, or, more specifically, about 5 mass % to about 20 mass %”, is inclusive of the endpoints and all intermediate values of the ranges of “about 5 mass % to about 25 mass %,” etc.). “Combination” is inclusive of blends, mixtures, alloys, reaction products, and the like. Furthermore, the terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item. The modifier “about” used in connection with a quantity is inclusive of the state value and has the meaning dictated by context, (e.g., includes the degree of error associated with measurement of the particular quantity). The suffix “(s)” as used herein is intended to include both the singular and the plural of the term that it modifies, thereby including one or more of that term (e.g., the reactor(s) includes one or more reactors). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and can or can not be present in other embodiments. In addition, it is to be understood that the described elements can be combined in any suitable manner in the various embodiments. As used herein, the terms sheet, film, plate, and layer, are used interchangeably, and are not intended to denote size.

While the invention has been described with reference to several embodiments thereof, it will be understood by those skilled in the art that various changes can be made and equivalents can be substituted for elements thereof without departing from the scope of the invention. In addition, many modifications can be made to adapt a particular situation or material to the teachings of the invention without departing from the essential scope thereof. Therefore, it is intended that the invention not be limited to the particular embodiments disclosed as the best mode contemplated for carrying out this invention, but that the invention will include all embodiments falling within the scope of the appended claims.

1. A method for pre-treating an oil, comprising:
introducing the oil, an alcohol, and an acid catalyst to a first continuous stirred tank reactor vessel, wherein the oil comprises greater than 0.5 mass % free fatty acids;
selectively reacting the oil and the alcohol to convert the free fatty acids to alkyl esters, wherein the reaction does not produce glycerol;
removing a pre-treated oil stream from a lower portion of the first continuous stirred tank reactor, wherein the pre-treated oil stream comprises the alkyl esters; and
recycling the alcohol and acid catalyst from an upper portion of the first continuous stirred tank reactor.

2. The method of claim 1, further comprising combining the oil with the alcohol and the acid catalyst to form a combined liquid stream before introduction to the first continuous stirred tank reactor.

3. The method of claim 2, wherein the alcohol and acid catalyst are recycled back to the combined liquid stream.

4. The method of claim 1, further comprising heating the oil, alcohol, and acid catalyst in the continuous stirred tank reactor.

5. The method of claim 1, wherein the oil is selected from the group consisting of vegetable oil, sunflower seed oil, soy bean oil, corn oil, cottonseed oil, almond oil, groundnut oil, palm oil, coconut oil, linseed oil, castor oil, rapeseed oil, industry tallow, abattoir, and combinations comprising at least one of the foregoing.

6. The method of claim 1, wherein the oil comprises used cooking oil.

7. The method of claim 1, wherein the alcohol is selected from the group consisting of C₁-C₄ alcohols, and combinations comprising at least one of the foregoing alcohols.

8. The method of claim 1, wherein the alcohol is selected from the group consisting of methanol, ethanol, isopropanol, butanol, multivalent alcohol, and combinations comprising at least one of the foregoing alcohols.

9. The method of claim 1, wherein the acid catalyst is a hydrochloric acid.

10. The method of claim 1, wherein a percent conversion of the free fatty acids to the liquid alkyl ester is greater than or equal to about 95 mass percent.

11. The method of claim 1, wherein the alcohol and acid catalyst are recycled and introduced to the first continuous stirred tank reactor vessel separately from the oil.

12. A method for pre-treating an oil, comprising:
combining the oil with an alcohol and a acid catalyst to form a combined liquid stream, wherein the oil comprises greater than 0.5 mass % free fatty acids;
introducing the combined liquid stream to a continuous stirred tank reactor vessel comprising a first inlet and a first outlet;
selectively reacting the oil and the alcohol to convert the free fatty acids to alkyl esters and produce a pre-treated oil, wherein the reaction does not produce glycerol;
separating the pre-treated oil from the alcohol and acid catalyst in a liquid/liquid separator vessel; wherein the pre-treated oil stream comprises the alkyl esters;
removing the pre-treated oil stream from a lower portion of the liquid/liquid separator vessel; and
recycling the alcohol and acid catalyst from an upper portion of the liquid/liquid separator vessel to the combined liquid stream.

13. The method of claim 12, wherein the combined liquid stream flows into the liquid/liquid separator in a laminar flow.

14. The method of claim 12, wherein the flow of the combined liquid stream into liquid/liquid separator has a Reynold's number of less than or equal to 2,100.

15. The method of claim 12, further comprising agitating the combined liquid within the continuous stirred tank reactor.

16. The method of claim 12, further comprising selectively reacting the oil and the alcohol in a plurality of continuous stirred tank reactors.

17. The method of claim 12, wherein the oil is selected from the group consisting of vegetable oil, sunflower seed oil, soy bean oil, corn oil, cottonseed oil, almond oil, groundnut oil, palm oil, coconut oil, linseed oil, castor oil, rapeseed oil, industry tallow, abattoir, and combinations comprising at least one of the foregoing.

18. A system for pre-treating an oil feed stream, comprising:
a first continuous stirred tank reactor comprising a combined liquid inlet, a first reaction stream outlet, and a first agitator, wherein the combined liquid inlet is in fluid communication with a liquid oil source comprising greater than 0.5 mass % free fatty acids and a liquid alcohol source comprising an acid catalyst, wherein the free fatty acids are configured to react with the alcohol source and be converted to alkyl esters; and
a liquid/liquid separator in fluid communication with the first continuous stirred tank reactor, wherein the separator is configured to separate the alkyl esters and the oil from the alcohol and the acid catalyst, wherein the separator comprises a separator inlet, a pre-treated oil outlet at a lower portion of the separator, and a recycle outlet at an upper portion of the separator, wherein the pre-treated oil outlet is configured to remove the oil and the alkyl esters and the recycle outlet is configured to remove the alcohol and the acid catalyst.

19. The system of claim 18, further comprising a plurality of continuous stirred tank reactors disposed in fluid communication between the first continuous stirred tank reactor and the liquid/liquid separator, wherein the plurality of continuous stirred tank reactors comprise an inlet, an outlet, and an agitator.

20. The system of claim 19, wherein the first reaction stream outlet is in fluid communication with a bypass line, wherein the bypass line is in direct fluid communication with the separator inlet.

21. The system of claim 20, wherein the bypass line is in further fluid communication with each of the plurality of continuous stirred tank reactor inlets and outlets.

22. The system of claim 18, further comprising an automated valve control system in operative communication with the oil source and the bypass line, wherein the automated valve control system comprises and a sensor and a controller and is configured to determine a concentration of the free fatty acids in the oil source and control a flow path of the oil through the bypass line based on the predetermined concentration.
